(19) United States
(12) Patent Application Publication
(10) Pub. No.: US 2021/0401934 A1
(43) Pub. Date: Dec. 30, 2021

(54) PROTEIN FUNCTIONALIZED HYALURONIC ACID COATED CHITOSAN NANOPARTICLE AND METHOD OF PREPARATION

(71) Applicant: KING ABDULAZIZ CITY FOR SCIENCE AND TECHNOLOGY, Riyadh (SA)

(72) Inventors: Afshan MASOOD, Riyadh (SA); Assim ALFADDA, Riyadh (SA); Ibrahim ALRADWAN, N/A (SA); Ibrahim ALANAZI, Riyadh (SA); Abdulaziz ALMALIK, Riyadh (SA); Ali AL-HASAN, Riyadh (SA); Hicham BENABDELAKMEL, Riyadh (SA); Waleed ALGHAMDI, Riyadh (SA); Haitham ALRABIAH, Riyadhl (SA)

(21) Appl. No.: 17/045,317

(22) PCT Filed: Apr. 9, 2018
(86) PCT No.: PCT/SA2018/000011

§ 371 Date: Oct. 5, 2020

Related U.S. Application Data
(60) Provisional application No. 62/483,515, filed on Apr. 10, 2017.

(30) Foreign Application Priority Data
Oct. 29, 2017 (SA) 117390079

(51) Int. Cl. A61K 38/17; A61K 9/16; A61K 47/69; A61K 47/61

(52) U.S. Cl. CPC A61K 38/1741; A61K 9/1676; A61K 47/61; A61K 47/6939; A61K 9/1652

(57) ABSTRACT

A protein functionalized anti-inflammatory nanoparticle and a method of preparing the protein functionalized anti-inflammatory nanoparticle is disclosed. The protein functionalized anti-inflammatory nanoparticle includes a central core comprising a hyaluronic acid coated chitosan nanoparticle and surface adsorbed anti-inflammatory proteins forming an outer shell around the central core, wherein the surface adsorbed anti-inflammatory protein is AGP (alpha-1-acid glycoprotein). The method of preparation includes dispersing chitosan nanoparticles in acetic acid/acetate buffer to produce a dispersion, adding an equal amount of acetate buffer containing hyaluronic acid under vigorous stirring to form hyaluronic coated chitosan nanoparticle (HA-CS) and functionalizing the hyaluronic coated chitosan nanoparticle with surface adsorbing anti-inflammatory protein AGP, to form the protein functionalized anti-inflammatory nanoparticle.

### FIELD OF INVENTION

The present invention generally relates to the field of surface functionalized nanoparticles. More specifically, the invention relates to a protein functionalized anti-inflammatory hyaluronic acid coated chitosan nanoparticle and method of preparation thereof.

### BACKGROUND OF INVENTION

Acute inflammation at a target area of a drug delivering system is a serious concern while designing nanoparticle systems which are employed in drug delivery systems Immunogenicity is an important parameter to be considered during the preparation of immune-suppressing nanoparticle systems as they are detrimental in the effective biocompatibility of the nanoparticle system. Also, uncontrolled immune stimulation, if unresolved, may lead to lethal consequences such as organ failures, which can be widespread and may further lead to mortality during conditions such as rheumatoid arthritis, stroke, hypovolemia, septic shock, sepsis, skin burns, trauma and likewise conditions.

Chitosan, a copolymer of β-(1→4)-linked D-glucose-2-amine and N-acetyl-D-glucose-2-amine, has been exhaustively employed in drug delivery systems. However, a cationic surface associated with chitosan nanoparticles, reduces the circulation time and bioavailability of the nanoparticle system in a biological environment. Now, while chitosan nanoparticles further associated with anionic polysaccharides, increase circulation time, rate of macrophage uptake is decreased. Therefore, chitosan nanoparticles associated with anionic polysaccharides require an association for further triggering phagocytosis.

Therefore, there exists a need for a biocompatible nanoparticle system with immune-suppressing properties to prevent/reduce acute inflammation and increase the blood circulation time.

### BRIEF DESCRIPTION OF DRAWINGS

The accompanying figures where like reference numerals refer to identical or functionally similar elements throughout the separate views and which together with the detailed description below are incorporated in and form part of the specification, serve to further illustrate various embodiments and to explain various principles and advantages all in accordance with the invention.

FIG. 1 is illustrative of an inflammatory focused nanoparticle-protein-function network, depicting a mapping of nineteen serum proteins (inflammatory-related molecular functions) adsorbed by chitosan nanoparticles (CS NPs), hyaluronic acid-coated CS NPs (HA-CS NPs), and alginate-coated CS NPs (Alg-CS NPs).

FIG. 2a is illustrative of the mass spectrometric analysis, thereby depicting overlap comparison of protein coronas formed by chitosan nanoparticles (CS NPs), hyaluronic acid-coated CS NPs (HA-CS NPs), and alginate-coated CS NPs (Alg-CS NPs) as controls. FIG. 2b is illustrative of a Venn diagram depicting the differences and similarities in the number of corona proteins identified between each nanoparticle system, respectively.

FIG. 3 is illustrative of mechanism of creating immune modulating nanomaterials, namely protein functionalized HA-CS NPs in accordance with the present invention.

FIG. 4 is illustrative of protein coating and quantification of bound proteins associated with varying doses of protein coating of AGP.

FIGS. 5a and 5b are illustrative of the size and zeta-potential distributions of chitosan nanoparticles (CS NPs), hyaluronic acid-coated CS NPs (HA-CS NPs), and alginate-coated CS NPs (Alg-CS NPs). FIG. 5c illustrates the statistical analysis of CS NPs, HA-CS NPs, and Alg-CS NPs including hydrodynamic diameter (Z-average size), polydispersity index (PDI), and zeta potential measurements.

FIG. 6a is illustrative of the comparative size distributions of chitosan nanoparticles (CS NPs), hyaluronic acid-coated CS NPs (HA-CS NPs), and alginate-coated CS NPs (Alg-CS NPs), performed before and after incubation with serum. FIG. 6b refers to the SDS-PAGE gel electrophoresis of CS NPs, HA-CS NPs, and Alg-CS NPs. FIG. 6c illustrates a bar graph depicting quantitative differences in the amount of corona proteins (μg/μL) adsorbed to each of the different NPs namely Alg-CS NPs, HA-CS NPs and CS NPs.

FIG. 7a is illustrative of a qualitative measurement of wound closure as a function of MDA-MB-231 cells migration rate. FIG. 7b is illustrative of a quantitative measurement of wound closures as a function of MDA-MB-231 cells migration rate.

### DETAILED DESCRIPTION

Before describing in detail embodiments that are in accordance with the invention, it should be observed that the embodiments reside primarily in a protein functionalized anti-inflammatory hyaluronic acid coated chitosan nanoparticle (HA-CS NPs) and a method of preparation thereof. The method of preparation includes functionalizing of the HA-CS NP with surface adsorbing anti-inflammatory proteins.

In this document, the terms “comprises,” “comprising,” or any other variation thereof, are intended to cover a non-exclusive inclusion, such that a process, method, article or composition that comprises a list of elements does not include only those elements but may include other elements not expressly listed or inherent to such process, method, article or composition. An element proceeded by “comprises . . . a” does not, without more constraints, preclude the existence of additional identical elements in the process, method, article or composition that comprises the element.

Various embodiments of the invention provide a protein functionalized anti-inflammatory HA-CS NP and a method of preparation thereof.

In accordance with the present invention, a protein functionalized anti-inflammatory HA-CS NP includes a central core and an outer shell surrounding the central core. The central core in accordance of the invention includes the HA-CS NP and the outer shell comprises functionalized surface adsorbed anti-inflammatory proteins, bound to the central core. The anti-inflammatory proteins functionalized to the HA-CS NP are ITIH4 (inter-alpha-trypsin inhibitor heavy chain 4) and AGP (alpha-1-acid glycoprotein, also known as orosomucoid). The central core and the functionalized anti-inflammatory particles undergo a chemisorption process, thereby the ITIH4 protein forming an ester bond between the carboxyl groups of the aspartates at their C′ termini and the C-6 hydroxyl groups of the HA. Furthermore, the AGP protein chemisorbed onto the HA layer forms a glycosidic bond through the glycan branches of AGP.

In one embodiment, a combination of anti-inflammatory proteins ITIH4 and AGP are bound to the central core HA-CS NP, forming stabilized nanoparticle systems by virtue of the strong covalent bonds formed between the anti-inflammatory proteins rendering a stable protein corona.

In another embodiment, the anti-inflammatory proteins bound to the central core may include either ITIH4 or AGP.

Referring to Table 1, Table 1 depicts the list of adsorbed proteins forming the protein coronas of the by CS NPs, HA-CS NPs, and Alg-CS NPs respectively, thereby listing the adsorbed proteins.

TABLE 1

| CS NPs | | HA-CS NPs | | Alg-CS NPs | |
|---|---|---|---|---|---|
| ID | Protein | ID | Protein | ID | Protein |
| G3X6N3 | Serotransferrin | G3X6N3 | Serotransferrin | G3X6N3 | Serotransferrin |
| E1BMJ0 | Uncharacterized | E1BMJ0 | Uncharacterized | E1BMJ0 | Uncharacterized |
| P02769 | Serum albumin | P02769 | Serum albumin | P02769 | Serum albumin |
| Q3ZBS7 | Uncharacterized | Q3ZBS7 | Uncharacterized | Q3ZBS7 | Uncharacterized |
| P34955 | Alpha-1- | P34955 | Alpha-1- | P34955 | Alpha-1- |
| | antiproteinase | | antiproteinase | | antiproteinase |
| | (SERPINA1) | | (SERPINA1) | | (SERPINA1) |
| Q7SIH1 | Alpha-2- | Q7SIH1 | Alpha-2- | Q7SIH1 | Alpha-2- |
| | macroglobulin | | macroglobulin | | macroglobulin |
| | (A2M) | | (A2M) | | (A2M) |
| P12763 | Alpha-2-HS- | P12763 | Alpha-2-HS- | P12763 | Alpha-2-HS- |
| | glycoprotein | | glycoprotein | | glycoprotein |
| | (AHSG) | | (AHSG) | | (AHSG) |
| A2I7M9 | Serpin A3-2 | A2I7M9 | Serpin A3-2 | A6QM09 | Uncharacterized |
| | (SERPINA3-2) | | (SERPINA3-2) | | |
| P56652 | Inter-alpha-trypsin | P56652 | Inter-alpha-trypsin | B0JYQ0 | ALB protein (ALB) |
| | inhibitor heavy chain | | inhibitor heavy chain | | |
| | H3 (ITIH3) | | H3 (ITIH3) | | |
| Q2KJF1 | Alpha-1B- | Q2KJF1 | Alpha-1B- | E1B726 | Plasminogen |
| | glycoprotein (A1BG) | | glycoprotein (A1BG) | | |
| Q3SZ57 | Alpha-fetoprotein | Q3SZ57 | Alpha-fetoprotein | E1BH06 | Uncharacterized |
| | (AFP) | | (AFP) | | |
| A5D7R6 | Inter-alpha-trypsin | F1MMD7 | Inter-alpha-trypsin | A5D7R6 | Inter-alpha-trypsin |
| | inhibitor heavy chain | | inhibitor heavy chain | | inhibitor heavy |
| | H2 (ITIH2) | | H4 (ITIH4) | | chain H2 (ITIH2) |
| G3N0V2 | Uncharacterized | G3N1Y3 | Uncharacterized | G3N0V2 | Uncharacterized |
| P02081 | Hemoglobin fetal | Q3SZR3 | Alpha-1-acid | P02081 | Hemoglobin fetal |
| | subunit beta (HBB) | | glycoprotein (AGP) | | subunit beta (HBB) |
| P17697 | Clusterin | F1MVK1 | Uncharacterized | P17697 | Clusterin |
| Q27984 | Alpha1- | | | Q28921 | Alpha 1- |
| | antichymotrypsin | | | | antichymotrypsin |
| | isoform pHHK12 | | | | (ACT) (Fragment) |
| | (Fragment) | | | | |
| F1N1W7 | Neural cell adhesion | | | Q95121 | Pigment |
| | molecule 1 (NCAM1) | | | | epithelium-derived |
| | (Fragment) | | | | factor (SERPINF1) |
| | | | | P00735 | Prothrombin |
| | | | | F1MJH1 | Gelsolin |
| | | | | Q28085 | Complement factor |
| | | | | | H (CFH) |
| | | | | Q1JPD0 | Complement C8 |
| | | | | Q9N2I2 | Plasma serine |
| | | | | | protease inhibitor |
| | | | | | (SERPINA5) |
| | | | | F1MSZ6 | Antithrombin-III |
| | | | | F1MLW8 | Uncharacterized |

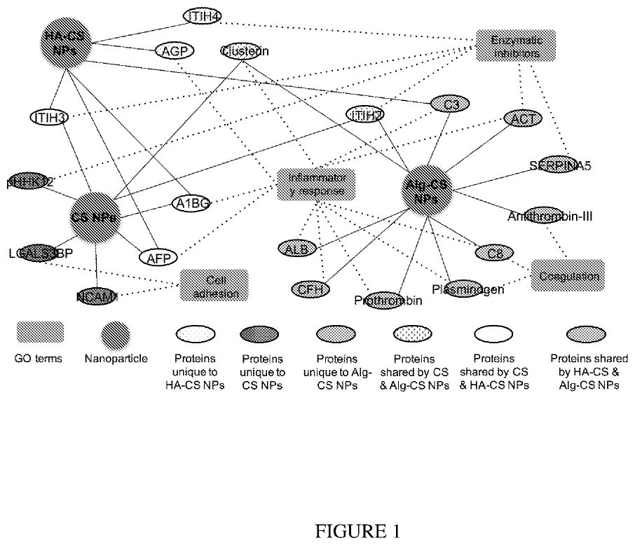

FIG. 1 illustrates an inflammatory focused protein nanoparticle protein function network, wherein the protein network includes a mapping of nineteen serum proteins (inflammatory-related molecular functions) adsorbed by CS NPs, HA-CS NPs, and Alg-CS NPs respectively, to establish a comparative study. Referring to FIG. 1, the spherical nodes in the protein network are representative of nanoparticles, the oval nodes are representative of the inflammatory proteins and the rectangular nodes are representative of molecular functions. Furthermore, the solid lines in the protein network are representative of proteins absorbed specific to each nanoparticle system, while the dotted lines represent the molecular functions related to the identified proteins. Based on the unique protein signature of HA-CS NPs, alpha-1-acid glycoprotein (AGP) and inter-alpha-trypsin inhibitor heavy chain H4 (ITIH4) are observed to be uniquely adsorbed proteins onto HA-CS NPs, absent from the protein coronas of both CS and Alg-CS NPs, thereby facilitating the formation of the protein functionalized anti-inflammatory HA-CS NP of the present invention.

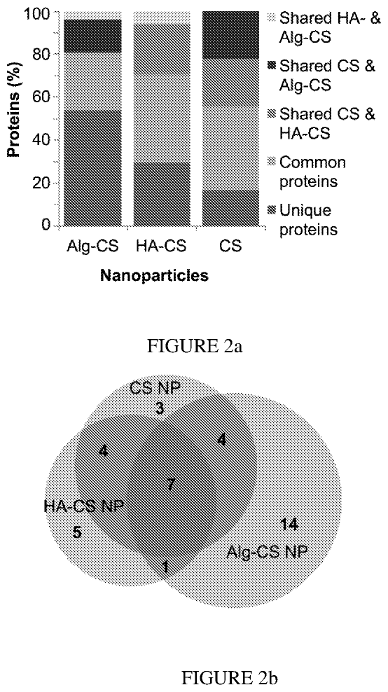

FIGS. 2a and 2b are representative of the comparison of protein coronas formed by CS NPs, HA-CS NPs, and Alg-CS NPs, depicting the differences and similarities (overlap) in the number of proteins in the protein corona of each of the three nanoparticles. The unique protein signatures in the protein corona of the three nanoparticles contributed to 17% (3 proteins), 31% (5 proteins), and 56% (14 proteins) of the distinctive proteins. Further referring to FIGS. 2a and 2b, although the degree of similarities between the protein coronas formed around the three nanoparticle systems identified seven common proteins adsorbed onto all three NPs, four common proteins shared by CS and Alg-CS NPs, another four common proteins were shared by CS and HA-CS NPs and one common protein was shared by HA-CS and Alg-CS NPs, two unique anti-inflammatory proteins was adsorbed onto HA-CS NPs. Also, both CS and Alg-CS NPs exclusively adsorbed clusterin, while it was not identified within the protein corona of HA-CS NPs, thereby facilitating formation of a low immunogenic nanoparticle system as clusterin is associated with various host immune processes including immune regulation, cell adhesion, and active cell death.

Hereinafter, a method of preparing the protein functionalized anti-inflammatory HA-CS NP is described.

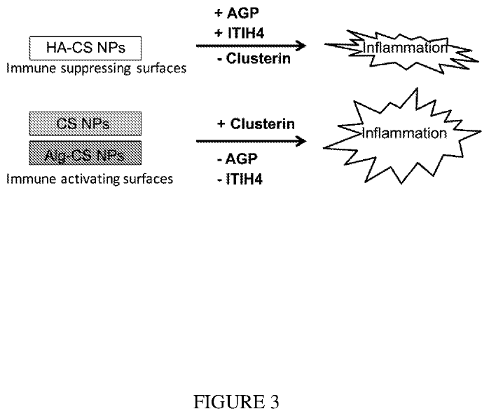

The method of preparation of the protein functionalized anti-inflammatory HA-CS NP begins with dispersing chitosan nanoparticles in 0.1 M acetic acid/acetate buffer at a pH of 5, to produce a dispersion. The ensuing step includes adding an equal amount of acetate buffer containing hyaluronic acid at a concentration of 1.5 mg/ml, under vigorous stirring for a time period of 30 minutes at 1200 rpm to form HA-CS, wherein the HA-CS NP is dialyzed against deionized water. Thereafter, in a crucial step, the HA-CS NP undergoes functionalizing with surface adsorbing anti-inflammatory proteins as illustrated in FIG. 3. The inflammatory proteins bound to the HA-CS NP is selected from either ITIH4 or AGP. In another embodiment, the central core is functionalized with a combination of ITIH4 and AGP.

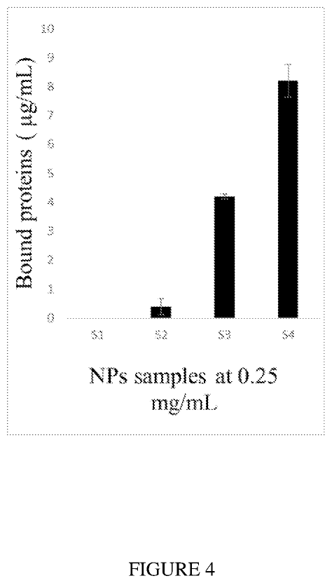

Functionalizing of the HA-CS NPs with surface adsorbing anti-inflammatory proteins includes a plurality of steps. More specifically, the functionalizing of the HA-CS NPs with surface adsorbing anti-inflammatory protein Alpha-1 Acid glycoprotein (AGP) includes dissolving the Alpha-1 Acid glycoprotein (AGP) in PBS buffer at a pH of 7.4, at a first step. In a next step, the dissolved Alpha-1 Acid glycoprotein (AGP) is mixed with 1 ml of HA-CS NPs at 0.25 mg/mL to reach a concentration of 100 μg/mL of AGP functionalized HA-CS NPs (AGP-HA-CS NP). Subsequently, HA-CS NPs in the presence of varying doses of AGP including 5, 10, 20, and 30 μg/ml were gently mixed to form varying suspensions, followed by vortexing for a time duration of 1 minute to obtain four homogenous suspensions of AGP-HA-CS NP, as illustrated in FIG. 4. FIG. 4 is illustrative of protein coating and quantification of bound proteins associated with varying doses of protein coating of AGP. Further, the four homogenous suspensions of AGP-HA-CS NP of varying doses of 5, 10, 20, and 30 μg/ml respectively, is transferred to a rotating platform at a temperature of 37° C. and at a speed of 50 rpm to facilitate the adsorption process for a time duration of 30 minutes of incubation period. After incubating the four homogenous suspensions of AGP-HA-CS NP, AGP-HA-CS NPs were separated from unbound AGP from the destabilized protein corona, by centrifugation at 15000 rpm at room temperature for a time duration of 15 minutes. A chemisorption process occurs, thereby forming ester and glycosidic bonds between the HA-CS NPs and through the glycan branches of AGP, thereby rendering bound protein (AGP) concentrations of 0, 0.4±0.3 μg/ml, 4.2±0.1 μg/ml and 8.2±0.6 μg/ml at initial concentrations of 5, 10, 20, and 30 μg/ml respectively, as depicted in Table 2 below.

TABLE 2

| | | bound |
|---|---|---|
| | Initial Cons. | proteins |
| NPs coated | (μg/mL) | (μg/mL) |
| S1 | 5 | 0 |
| S2 | 10 | 0.4 ± 0.3 |
| S3 | 20 | 4.2 ± 0.1 |
| S4 | 30 | 8.2 ± 0.6 |

The method of preparing CS NPs further includes numerous steps. A first step of preparation of chitosan particle includes dissolving chitosan at a concentration of 0.07% weight, in 4.6 mM of HCl. The pH of the chitosan solution was then adjusted to 5 by adding appropriate volumes of 0.1 M NaOH and further underwent continuous stirring overnight. In the ensuing step, 0.1% weight, Triphenyl phosphate solution (TPP) in deionized water was prepared and the pH of the solution was adjusted to 5 using 0.1 M HCl. The prepared TPP solution was added to the chitosan solution in a mass ration of 1:9, to prepare a complexation solution. Considering an example, 0.214 mL of TPP solution was added to 2.786 mL of CS solution, where the final concentrations of TPP and CS represented 0.0071 and 0.064% weight respectively, thereby resulting in a 1:9 mass ratio of TPP:CS. All solutions were filtered through a 0.22 μm pore size filter. The complexation solution further undergoes magnetic stirring and agitation, for a time period of 30 min at room temperature. Finally, the complexation solution is left undisturbed for a period of 24 hours after a brief sonication to produce CS NPs. The dispersed CS NPs were then dialyzed against deionized water (MWCO 1000 kDa).

Characterization Studies:

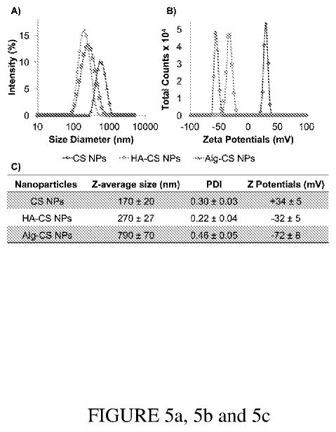

As illustrated in FIGS. 5a and 5b, CS NPs, HA-CS NPs, and Alg-CS NPs prepared using methodologies known in the art are characterized from the point of view of size and zeta-potential distributions. The characterization included characterization in buffers followed by characterization in serum for biological molecule interaction, wherein CS NPs showed large and positive zeta potentials, and upon coating with anionic HA, the positive charge of CS NPs was retained and significant increase in size diameter ranging between 170 nm to 270 nm was observed. Further, Alg-CS NPs showed higher negative zeta potential and three times larger size (in diameters) in comparison with CS NPs, HA-CS NPs. FIG. 5c illustrates the statistical analysis of CS NPs, HA-CS NPs, and Alg-CS NPs including hydrodynamic diameter (Z-average size), polydispersity index (PDI), and zeta potential measurements performed at room temperature using a Zetasizer Nano ZS instrument (Model ZEN3600, Malvern Instruments Ltd., UK) fitted out with a solid state HeNe laser (λ=633 nm) at a scattering angle of 173°.

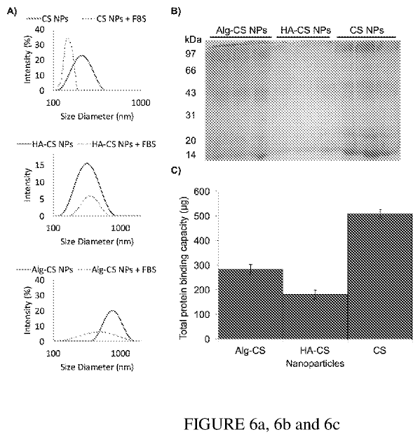

Further, moving to FIG. 6a, comparative size distributions of CS NPs, HA-CS NPs, and Alg-CS NPs, was performed before and after incubation with serum. The CS NPs after incubation with serum to form protein coronas included a significantly lower size distribution due to rapid agglomeration of nanoparticles followed by sedimentation. The HA-CS NPs was observed to have insignificant changes in terms of size distribution after incubation with serum. The size distribution of Alg-CS NPs was observed to be reduced in comparison with the size distribution before the incubation with serum. FIG. 6b refers to the SDS-PAGE gel electrophoresis of CS NPs, HA-CS NPs, and Alg-CS NPs. On performing SDS-PAGE gel electrophoresis, no significant changes in size distribution of HA-CS NPs was observed after incubation with serum. Furthermore, the gel electrophoresis bands of HA-CS NPs were pale and barely detectable in comparison with the CS NPs and Alg-CS NPs, owing to the significantly decreased protein adsorption. FIG. 6c illustrates a bar graph depicting quantitative differences in the amount of corona proteins (μg/μL) adsorbed to each of the different NPs namely Alg-CS NPs, HA-CS NPs and CS NPs. Net charge density of serum proteins is negative at physiological pH and particles with cationic surfaces exhibit a rapid and intense adsorption of high affinity and highly abundant proteins upon exposure to biological environments, which results in a dense but less diverse protein corona, thereby CS NPs showing highest reactivity to serum proteins.

In an embodiment, the protein functionalized anti-inflammatory HA-CS NPs, specifically AGP-HA-CS NPs is employed in suppressing the immunity of activated cancer cells. AGP-HA-CS NPs is added to activated breast cancer cells (MDA-MB-231), with activated migration by virtue of LPS (Lipopolysaccharide). LPS (Lipopolysaccharides) enables accelerated migration of cancer cells, thereby acting as a stimulated immune response. The activated breast cancer cells are grown to near confluency and further scratched to create a wound.

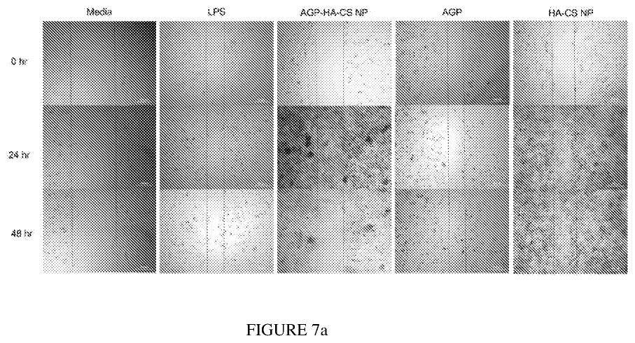

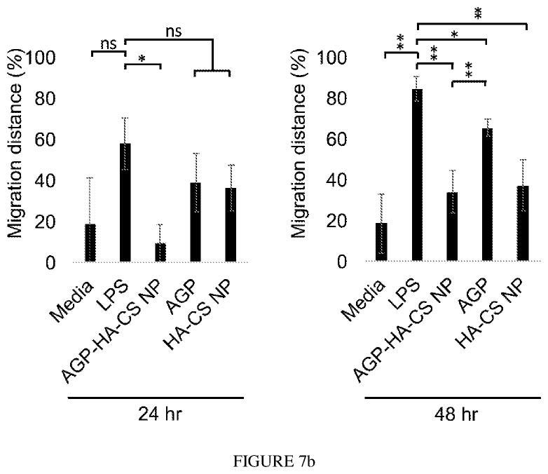

FIG. 7a illustrates the qualitative measurements of wound closures as a function of MDA-MB-231 cells migration rate. Further referring to FIG. 7a, width of the created wound was monitored for a time duration of 48 hours to determine wound closure rate as a function of activator LPS and the immune inhibitory effects of AGP-HA-CS NPs. FIG. 7a is indicative of a low number of cells migrating toward the wound area in the presence AGP-HA-CS NPs and LPS, similar to the cell migration behavior in the presence of inactivated breast cancer cells. FIG. 7a thereby illustrates a slow wound closure rate in the presence of AGP-HA-CS NPs and LPS, similar to a wound closure rate in the presence of inactivated breast cancer cells acting as a negative control. FIG. 7a illustrates a high number of cells migrated within the wound area in the presence of LPS and HA-CS NPs or AGP, thereby ruling out the effect of the uncoated HA-CS NPs and free AGP as an efficient immune suppressant. Moving to FIG. 7b that illustrates the quantitative measurements of wound closures as a function of MDA-MB-231 cells migration rate, wherein significant migration inhibition indicative of significant immune suppressions, exerted by AGP-HA-CS NPs when compared to free AGP and HA-CS NPs was observed, especially in the first 24 hours. AGP, therefore is required to be adsorbed onto NPs for enhanced immune suppression as opposed to free AGP. Further observations include failure of free AGP as well as uncoated HA-CS NPs to rescue breast cancer cells from the stress caused by LPS. Again, referring to FIG. 7b, incubation for 48 hrs included the effect of AGP-HA-CS NPs with higher efficacy when compared to free AGP.

In another embodiment, the protein functionalized anti-inflammatory HA-CS NP with surface adsorbed proteins AGP is employed in a drug delivery system. Considering an example, a widely known immune suppressant corticosteroid, namely dexamethasone (DXM) is delivered by the protein functionalized HA-CS NP in accordance with the present invention to reduce acute inflammatory responses in conditions like rheumatoid arthritis, stroke, hypovolemia, septic shock, sepsis, skin burns, trauma and likewise.

Those skilled in the art will realize that the above recognized advantages and other advantages described herein are merely exemplary and are not meant to be a complete rendering of all of the advantages of the various embodiments of the invention.

In the foregoing specification, specific embodiments of the invention have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of the invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

1. A protein functionalized anti-inflammatory hyaluronic acid coated chitosan nanoparticle (HA-CS NP).

2. The protein functionalized anti-inflammatory hyaluronic acid coated chitosan nanoparticle (HA-CS NP) as claimed in claim 1, comprising:
a central core hyaluronic acid coated chitosan nanoparticle; and
surface adsorbed anti-inflammatory proteins forming an outer shell around the central core, wherein the surface adsorbed anti-inflammatory protein is AGP.

3. The protein functionalized anti-inflammatory HA-CS NP as claimed in claim 2, wherein the central core HA-CS NP has an average particle size ranging from 170 to 270 nanometers.

4. The protein functionalized anti-inflammatory HA-CS NP as claimed in claim 1, having a protein corona of the functionalized anti-inflammatory biocompatible nanoparticle with low immunogenicity.

5. The protein functionalized anti-inflammatory HA-CS NP as claimed in claim 1, which is employed as a nano-coating in a nano drug delivering medical device.

6. The protein functionalized anti-inflammatory HA-CS NP as claimed in claim 1, which is employed as a nano-coating in a medical device.

7. The protein functionalized anti-inflammatory HA-CS NP as claimed in claim 1, which is employed in suppressing the immunity of activated cancer cells.

8. The protein functionalized anti-inflammatory HA-CS NP as claimed in claim 1, which is employed in suppressing the immunity resulting from conditions selected from a group of conditions comprising of rheumatoid arthritis, stroke, hypovolemia, septic shock, sepsis, skin burns, and trauma.

9. A method of preparing functionalized anti-inflammatory biocompatible nanoparticle, the method comprising:
dispersing chitosan nanoparticles in 0.1 M acetic acid/acetate buffer at a pH of 5, to produce a dispersion;
adding an equal amount of acetate buffer containing hyaluronic acid under vigorous stirring for a time period of 30 minutes at 1200 rpm to form hyaluronic coated chitosan nanoparticle (HA-CS NP); wherein the HA-CS NP is dialyzed against deionized water; and
functionahzing the HA-CS NP with surface adsorbing anti-inflammatory proteins selected from a group of ITIH4, AGP and a combination of ITIH4 and AGP.

10. The method as claimed in claim 9, wherein the method includes functionalizing the HACS NP with surface adsorbing anti-inflammatory protein Alpha-1 Acid glycoprotein (AGP) further comprises:
dissolving the Alpha-1 Acid glycoprotein (AGP) in PBS at a pH adjusted to 7.4;
mixing dissolved Alpha-1 Acid glycoprotein (AGP) with 1 ml of HA-CS NPs at 0.25 mg/mL to reach a concentration of 100 μg/mL of AGP functionalized HA-CS NPs (AGP-HA-CS NP) suspension;
vortexing the (AGP-HA-CS NP) suspension to obtain a homogenous suspension of (AGP-HA-CS NP);
transferring homogenous suspension of (AGP-HA-CS NP) to a rotating platform at a temperature of 37° C. and at a speed of 50 rpm for a time duration of 30 minutes of incubation period to obtain incubated homogenous suspension of (AGP-HA-CS NP); and
centrifuging the incubated homogenous suspension of (AGP-HA-CS NP) at 15000 rpm at room temperature for a time duration of 15 minutes for separating AGP-HA-CS NPs from unbound AGP.

11. The method as claimed in claim 9, wherein the method includes preparing chitosan nanoparticles, the method of preparing chitosan nanoparticles comprising:
dissolving chitosan in 4.6 mM HCl adjusted to a pH of 5 to produce a 0.07% weight chitosan solution, wherein the chitosan solution undergoes continuous stirring;
adding TPP solution to the chitosan solution at a mass ratio of 1:9 to form a solution complexation, wherein the TPP solution is prepared in deionized water adjusted to a pH of 5;
magnetic stirring and agitating of solution complexation for a time period of 30 minutes at room temperature; and
sonicating the solution complexation to produce chitosan nanoparticles.

12. The method as claimed in claim 9, wherein adding acetate buffer containing hyaluronic acid includes hyaluronic acid at a concentration of 1.5 mg/ml.

13. The method as claimed in claim 9, wherein dissolving chitosan further includes dispersing chitosan at a concentration of 0.025 percentage weight.
